(19) United States
(12) Patent Application Publication
(10) Pub. No.: US 2015/0005234 A1
(43) Pub. Date: Jan. 1, 2015

(54) SOFT TISSUE IMPLANT

(71) Applicant: Amit Prakash Govil, Carlsbad, CA (US)

(72) Inventor: Amit Prakash Govil, Carlsbad, CA (US)

(21) Appl. No.: 14/320,885

(22) Filed: Jul. 1, 2014

Related U.S. Application Data
(60) Provisional application No. 61/841,601, filed on Jul. 1, 2013.

(51) Int. Cl. A61K 38/18; A61K 45/06

(52) U.S. Cl. CPC A61K 38/1866; A61K 38/1825; A61K 38/1841; A61K 45/06
USPC 514/8.1; 604/218

(57) ABSTRACT

Provided herein are soft tissue implants, methods of making, use, and administration thereof. The soft tissue implants can be prepared by harvesting cells or tissue from a donor and selectively lysing the cells or tissue to obtain the intracellular content. Also provided herein are delivery devices for delivering the soft tissue implants described herein and kits that include the soft tissue implants described herein.

### CROSS-REFERENCE TO RELATED APPLICATIONS

This application claims the benefit of U.S. provisional patent application Ser. No. 61/841,601 filed on Jul. 1, 2013, having the title “Soft Tissue Implant,” the disclosure of which is incorporated herein in its entirety.

### BACKGROUND

Changes in soft tissues occur as a result of the natural aging process as well as traumatic events, such as surgery, disease, or other conditions. Changes in these soft tissues can create undesirable soft tissue defects. For example, as aging occurs, loss of adipose and other soft tissues in the face results in wrinkles. Additionally, inflammation and fibrous tissue formation can occur after the addition of any type of implant in response to a foreign body being present.

In such instances, soft tissue implants are desirable to address some of the deleterious consequences of soft tissue changes. Current methods of obtaining soft tissue for the basis of a soft tissue implant rely on methods that remove several important cellular components, including key proteins, from the soft tissue implant after harvest. As such, there exists a need for improved soft tissue implants as well as methods of making soft tissue implants.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is a flow diagram illustrating embodiments of a method for harvesting soft tissue cells and retaining endogenous intracellular components.

FIG. 2 is a flow diagram illustrating embodiments of a method of incorporating the stored or un-stored slurry of FIG. 1 into a carrier substrate.

FIG. 3 is a flow diagram illustrating embodiments of a method of incorporating the stored or un-stored slurry of FIG. 1 into a soft tissue graft.

FIG. 4 shows one embodiment of a delivery device containing a slurry as produced according to the methods described herein.

FIG. 5 shows another embodiment of a delivery device containing a slurry as produced according to the methods described herein.

FIG. 6 demonstrates increased growth factor content in a carrier substrate combined with adipose-derived intracellular compounds (LipoAmp) as compared to control.

FIG. 7 shows in vivo implantation volume of a carrier substrate combined with adipose-derived intracellular compounds (LipoAmp) over time as compared to donor matched control implants.

FIGS. 8A and 8B demonstrate control staining (FIG. 8A) and hematoxylin and eosin staining (FIG. 8B) demonstrating ectopic adipogensis at the site of implantation of a carrier substrate containing adipose-derived intracellular compounds (LipoAmp).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, physiology, modern surgical techniques, microbiology, nanotechnology, organic chemistry, biochemistry, botany and the like, which are within the skill of the art. Such techniques are explained fully in the literature.

Definitions

In describing the disclosed subject matter, the following terminology will be used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within .+−.10% of the indicated value, whichever is greater.

As used herein, “effective amount” is an amount sufficient to effect beneficial or desired results. An effective amount can be administered in one or more administrations, applications, or dosages.

As used herein, “therapeutic” refers to treating or curing a disease or condition.

As used herein, “preventative” refers to hindering or stopping a disease or condition before it occurs or while the disease or condition is still in the sub-clinical phase.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “isolated” means separated from constituents, cellular and otherwise, with which the polynucleotide, peptide, polypeptide, protein, antibody, or fragments thereof, are normally associated in nature. A non-naturally occurring polynucleotide, peptide, polypeptide, protein, antibody, or fragments thereof, does not require “isolation” to distinguish it from its naturally occurring counterpart.

As used herein, “diluted” used in reference to an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “specific binding” refers to binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, “control” is an alternative subject or sample used in an experiment for comparison purposes and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “culturing” refers to maintaining cells under conditions in which they can proliferate and avoid senescence as a group of cells. “Culturing” can also include conditions in which the cells also or alternatively differentiate.

As used herein, “synergistic effect,” “synergism,” or “synergy” refers to an effect arising between two or more molecules, compounds, substances, factors, or compositions that is greater than or different from the sum of their individual effects.

As used herein, “additive effect” refers to an effect arising between two or more molecules, compounds, substances, factors, or compositions that is equal to or the same as the sum of their individual effects.

As used herein, “autologous” refers to being derived from the same subject that is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenically.

As used herein, “syngeneic” refers to subjects or donors that are genetically similar enough so as to be immunologically compatible to allow for transplantation, grafting, or implantation.

As used herein, “implant” or “graft,” as used interchangeably herein, refers to cells, tissues, or other compounds, including metals and plastics, that are inserted into the body of a subject.

As used herein, “filler” refers to a substance used to fill a cavity or depression. The filler can fill the depression such that it is level with the surrounding area or that the cavity is filled, such that the depth of the depression or volume of the cavity is decreased, or such that the area that was the depression is now raised relative to the areas immediately surrounding the depression.

As use herein, “immunogenic” or “immunogenicity” refers to the ability of a substance, compound, molecule, and the like (referred to as an “antigen”) to provoke an immune response in a subject.

As used herein, “exogenous” refers to a compound, substance, or molecule coming from outside a subject or donor, including their cells and tissues.

As used herein, “endogenous” refers to a compound, substance, or molecule originating from within a subject or donor, including their cells or tissues.

As used herein, “bioactive” refers to a material, compound, or other molecule that interacts with or causes an effect on any cell or tissue or other biological pathway in a subject.

As used herein, “physiological solution” refers to a solution that is about isotonic with tissue fluids, blood, or cells.

As used herein, “donor” refers to a subject from which cells or tissues are derived.

As used herein, “slurry” refers to the resultant product from any of the methods described herein. Accordingly, the slurry can be in any form resulting from the processing described herein, including but not limited to, dehydrated slurry or tissue, paste, powder, solution, gel, putty, particulate and the like.

As used herein, “extra cellular matrix” refers to the non-cellular component surrounding cells that provides support functions to the cell including structural, biochemical, and biophysical support, including but not limited to, providing nutrients, scaffolding for structural support, and sending or responding to biological cues for cellular processes such as growth, differentiation, and homeostasis.

As used herein, “complete extracellular matrix” refers to extracellular matrix that has all components (proteins, peptides, proteoglycans, and the like) present and may or may not include other cells that are embedded in the extra cellular matrix.

As used herein, “decellularized extracellular matrix” refers to complete extracellular matrix that has been processed to remove any cells embedded within the extracellular matrix.

As used herein, “extracellular matrix component” refers to a particular component. By way of a non-limiting example, an extracellular matrix comportment can be a specific class of comments (e.g. proteoglycans) or individual component (e.g. collagen I) that is separated or isolated from the other extracellular components. These components can be made synthetically.

As used herein “hydrogel” refers to a network of hydrophilic polymer chains that are dispersed in water. “Hydrogel” also includes a network of hydrophilic polymer chains dispersed in water that are found as a colloidal gel.

As used herein “self-assembling peptides” refer to peptides which undergo spontaneous assembly into ordered nanostructures. “Self-assembling peptides” include di-peptides, lego peptides, surfactant peptides, molecular paint or carpet peptides, and cyclic peptides.

Discussion

While soft tissue implants and grafts have many applications, current methods used to harvest and prepare the soft tissues for implantation are relatively crude and harsh and, importantly, result in the loss of key proteins and other molecules. In a typical allograft harvesting and processing procedure, a donor is prepped according to standard surgical procedures and the various tissues desired are recovered by surgical staff. Recovered tissues, which are the tissue grafts, are typically cultured prior to further processing to determine the level of bacterial contamination. Some tissues can be maintained in culture to retain the tissue's viability.

If, after culture, the soft tissue implant/graft is positive for a virulent organism, including but not limited to, Clostridia species, enterococci, or fungi, the tissue graft is discarded. However, this culture method is not completely reliable in determining bacterial contamination. Other tests on the donor, such as blood tests for HIV, hepatitis B and C, and syphilis are performed to determine the safety of the harvested allograft(s). Even these methods are not completely reliable.

As such, the allografts are typically further sterilized to reduce the microorganism contamination to less than about 10⁻³ microorganisms. Typical sterilization methods include, but are not limited to, combinations of washing with or without pressurization, centrifugation with various chemicals such as alcohols and/or detergents, and combining antibiotics with low-dose radiation. While these processing methods reduce the amount of microorganism contamination, they also can damage the tissue graft and result in the loss of many intracellular proteins and molecules.

On the one hand, the removal of intracellular proteins and molecules is good insofar as it reduces the immunogenicity of the allograft. Immogenicity is reduced because immunogenic extracellular components (e.g. proteins, lipoproteins, and other immunogenic molecules that reside in/on the cell membrane) are washed away during the stringent washing steps, which typically include lysing of the cells. However, the washing and lysing also results in the loss of the intracellular components of the cell (e.g. proteins, DNA, RNA, peptides, and other molecules that are contained within the cell). The loss of some of these endogenous intracellular components, such as growth factor proteins, can adversely affect the performance of the allograft and its incorporation into the surrounding tissue. Allografting of intact cells or tissue grafts that are not acellular is not successful due to the immunogenicity of the intact cells and cellularized tissues. These allografts are rarely successful and typically require that the recipient take immunosuppressants to maintain the allograft.

With these problems and limitations of current methods for preparing soft tissue implants and grafts in mind, the present disclosure provides methods of preparing soft tissue implants where the immunogenic portion of the cells are removed and at least a portion of the intracellular components are retained and processed into a soft tissue implant. The methods described herein are particularly suited for processing harvested adipose tissue and cells, as well as in vitro cultured adipose tissue and cells. Specifically, the methods described herein allow for collection of endogenous intracellular components of adipose cells and incorporate these components into soft tissue implants, grafts, and fillers for many reconstructive and surgical repair techniques.

In an embodiment, a soft tissue implant contains a bioactive intracellular component of an adipose cell and a carrier substrate, where the soft tissue implant is prepared by harvesting an adipose cell from a donor, selectively lysing the adipose sell to obtain the bioactive intracellular components and combining the bioactive intracellular component with a carrier substrate. In some embodiments, the soft tissue implant can be directly administered to a subject in need thereof.

In other embodiments, the soft tissue implant is a first soft tissue implant that is applied to a second soft tissue implant. The first soft tissue implant can be applied to a second soft tissue implant while the second soft tissue implant is outside the recipient of the second soft tissue implant (ex vivo). In other embodiments, the first soft tissue implant can be applied to the second soft tissue implant after the second soft tissue implant is already implanted in the recipient (in situ).

Accordingly, also provided are soft tissue implants, grafts, and fillers produced by the methods described herein. Also provided are devices for containing and/or delivering the soft tissue implants, grafts, and fillers produced by the methods described herein and kits containing the soft tissue implants, grafts, fillers and/or devices described herein. The methods, soft tissue implants, grafts, fillers, devices, and kits described herein offer several advantages to current soft tissue grafts at least insofar as they incorporate endogenous intracellular components, while minimizing the immunogenicity of the soft tissue implant.

Other compositions, compounds, methods, devices, systems, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

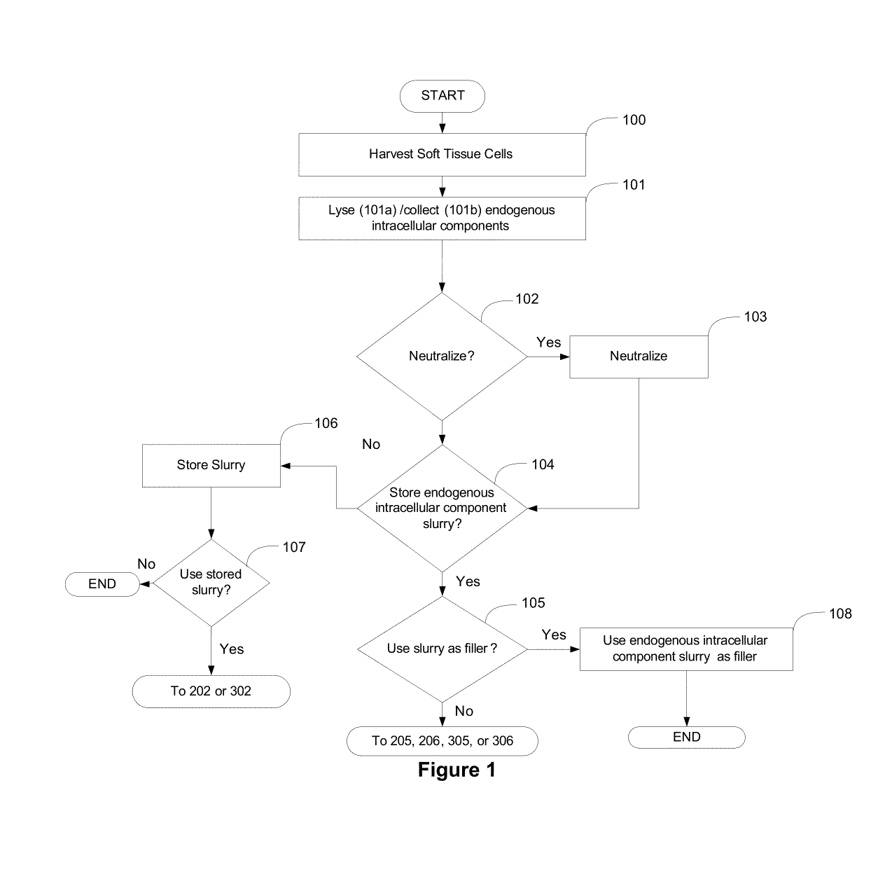

Discussion of the disclosed embodiments begins with FIG. 1, which is a flow diagram illustrating an embodiment of a method for harvesting soft tissue cells, particularly adipose cells, and collecting one or more of the endogenous intracellular components. In short, the method involves harvesting an adipose cell from a donor, selectively lysing the adipose cell to obtain a bioactive intracellular component and combining the bioactive intracellular component with a carrier substrate to form a combined bioactive intracellular component-carrier substrate. In some embodiments, the combined bioactive intracellular component-carrier substrate is administered to a subject in need thereof. The methods described herein produce a soft tissue implant containing a bioactive intracellular component of an adipose cell.

The method begins in an embodiment by harvesting cells from soft tissues from a donor or from an in vitro cell or tissue culture by a suitable method 100. Suitable harvesting methods are generally known in the art and include, but are not limited to, aspiration, scraping, dissection, and other surgical techniques known in the art. In one embodiment, tissue is excised in a desired shape and amount as determined by a medical practitioner. Factors that determine the shape and amount of the tissue to be excised include the physiological condition of the donor tissue and size of graft needed. In some embodiments, the tissue or cells are harvested at ambient temperature. In other embodiments, the tissue or cells are harvested at a temperature less than ambient temperature. In further embodiments, the tissues or cells are harvested at temperatures as low as about −210° C.

In embodiments, tissue can be minced, cut, ground, and/or chopped into particulates. In some of these embodiments, the particulates are about 1.5 times longer in one plane than another plane. In some embodiments, the elongated shape of these particulates may improve incorporation of the implant into surrounding tissue, remodeling of surrounding tissue, and tissue growth upon implantation. This may be due to an increase in surface area of the elongated implant particulates, which may facilitate vascularization.

Cutting, mincing, and grinding can further aid in separating the tissue into different constituents to further ease separation from the tissue, which allows for separation of the constituents based on density. In some embodiments, to obtain a specific constituent of tissue (e.g. adipose or collagen), the harvested tissue is cut, minced, ground, or otherwise mechanically manipulated and the constituents are separated out based on their density. In some embodiments, adipose tissue or cells are obtained from within another tissue (e.g. muscle) by this process. The profile of intracellular contents of cells can vary based on the environment in which the cell resides. Therefore, in some embodiments, the adipose cells are derived from intertissue (within or interspersed within another tissue) adipose tissue, as opposed to interstitial adipose tissue that is not interspersed within another tissue in order to obtain a particular intracellular content profile in the final implant product.

Soft tissues include, any tissue or organ that is not bone, including, but not limited to adipose tissue, muscle, cartilage, tendons, and ligaments. In one embodiment, the harvested cells are adipose cells. The soft tissues can be autologous, allogeneic, xenogeneic, or syngeneic in origin. In order to minimize immunogenicity, the use of autologous cells is most advantageous. In other words, it is preferred if the harvested cells were obtained directly or indirectly (i.e. from an in vitro culture containing cells from the subject to receive the implant) from the subject that is to receive the soft tissue implant. In an embodiment, autologous adipose cells are harvested. In other embodiments, the tissue or cells are allogeneic.

As previously mentioned, in some embodiments, the harvested soft tissue cells are cultured in vitro for an amount of time using suitable cell culture methods generally known in the art. One having ordinary skill will appreciate that the culture conditions will vary depending on the cell type. In some embodiments, cells from adipose tissue are cultured in vitro for about 1 day to about 6 months. In some embodiments, the cultured cells are harvested 100 as previously described. In an embodiment, adipose cells are harvested from a donor and cultured in vitro, until harvested 100 as previously described.

In some embodiments, the harvested cells are suspended in a physiological solution. Suitable physiological solutions include, but are not limited to, saline (about 0.9% w/v), phosphate-buffered saline, Ringer's solution, Tris-buffered saline, and HEPES (2-[4-(2-hydroxyethyl)piperazin-1-yl]ethanesulfonic acid)-buffered saline. In some embodiments, the concentration of harvested cells in the physiological solution ranges from about 1×10² cells/mL to about 1×10¹⁰ cells/mL.

Next, in some embodiments, the harvested cells are lysed 101a to release the endogenous intracellular components. After cell lysis, a cell lysate is generated, which contains the lysed cell membrane, intracellular contents, the physiological solution (if present), and the solution used to lyse the cells. The intracellular components include, but are not limited to, proteins (including enzymatic proteins and non-enzymatic proteins), protein complexes, nucleic acids, lipids, fatty acids, amino acids, peptides, simple sugars, carbohydrates, minerals, vitamins, ions (e.g. potassium, sodium, chloride, bicarbonate, magnesium, and calcium), hormones, and growth factors (which can be proteins or other types of molecules or macromolecules themselves). Examples of intracellular components include, but are not limited to aFGF, bFGF, VEGF, TGFB1, ANG, IGF, and the like. Lysing can occur by mechanical, chemical, and/or biological processes. Mechanical process include, but are not limited to, thermolysis, microfluidics, ultrasonics, electric shock, blending, milling, beadbeating, homogenization, french press, impingement, applying excessive shear, pressure, or vacuum forces, or combinations thereof.

For some embodiments, thermolysis includes freezing, freeze-thaw cycles, and heating to disrupt cell membranes. In other embodiments, microfluidics includes osmotic shock or crenation. Ultrasonic methods of lysis include, but are not limited to, sonications, sonoporation, sonochemistry, sonoluminescence, and sonic cavitation. Electric shock methods of lysis include, but are not limited to, electroporation and exposure of the cells to high voltage and amperage sources. Milling or beadbeating methods of cell lysis involve physically colliding or grinding the cells with an object or one another, in order to break the cell membranes. In some embodiments, excessive shear pressure is induced by aggressive pipetting through a small aperture centrifuging at a high rpm which results in a high gravitational force being applied to the cell, turbulent flow, or applying a vacuum to the cells, such that that the cell membranes are sheared apart.

In other embodiments, chemical methods are employed to lyse the cells. In some of these embodiments, cells are lysed after exposure to detergents, solvents, surfactants, hemolysis, or combinations thereof. Exposure to detergents and/or solvents may also disrupt cell membranes and remove lipid barriers surrounding the cells. Further, exposure to detergents, surfactants, and hemolysins can also aid in the removal of other debris that may be present in the cell solution. In other embodiments, cells are lysed due to a pH imbalance induced by exposure to an acidic (pH less than 7), basic (pH greater than 7) or neutral solution (pH equals 7). In additional embodiments, additional ions, such as sodium, potassium, and calcium, are added to the physiological solution to alter the osmolarity of the solution such that it is no longer isotonic. Examples include, but are not limited to, water, triton, peroxides, antibiotics, and other bioburden reducing solutions.

In further embodiments, the cells are lysed using a biological method or process. In some embodiments, the cells are contacted with an enzyme, such as lysozyme, mannases, proteases, lipidases, glycanases, or combinations thereof, which lyse the cell membranes. In other embodiments, viruses are employed to lyse the cell membranes.

Continuing with FIG. 1, as the endogenous intracellular components are released, at least some are collected 101b. In some embodiments, substantially all of the intracellular components are separated from the cell membrane components and collected. In other embodiments, a subset of the intracellular components is collected. In these embodiments, the desired intracellular components are collected and separated from the rest of the cell membrane fragments and/or the other intracellular components using a suitable separating technique. In these embodiments, where a selective subset of intracellular components is obtained during lysis, the steps 101a and 101b are collectively referred to as selective lysis. In some embodiments, the separated intracellular components are used in subsequent steps of the methods described herein. In other embodiments, the remaining intracellular components in the lysate are used in subsequent steps of the methods described herein. In either case, the portion containing the desired intracellular components is referred to as the endogenous intracellular component slurry in the remainder of the steps.

In some embodiments, the desired intracellular components are separated using a chromatography technique. Suitable chromatography techniques include, but are not limited to, size exclusion chromatography, ion exchange chromatography, expanded bed absorption chromatography, affinity chromatography (including but not limited to supercritical fluid chromatography), displacement chromatography, gas chromatography, liquid chromatography, column chromatography, planar chromatography (including, but not limited to paper chromatography, thin-layer chromatography), reverse-phase chromatography, simulated moving-bed chromatography, pyrolysis gas chromatography, fast protein liquid chromatography, high performance liquid chromatography, ultra high performance liquid chromatography, countercurrent chromatography, and chiral chromatography.

In other embodiments, the desired intracellular components are separated using an immunoseparation technique. In these embodiments, antibodies specific for a particular intracellular component are employed to bind the desired intracellular component. The antibody-intracellular component complex can then be separated from the rest of the lysate using antibody purification methods known in the art. In some embodiments, the antibody-intracellular component complex is separated from the lysate by exposing the lysate to an immunoglobulin affinity column. In other embodiments, the antibody is complexes to a magnetic compound or ion. In these embodiments, the antibody-intracellular component complex is separated from the complex using a magnetic field. After separation from the lysate, the antibody can be separated from the intracellular component using techniques generally known in the art.

In other embodiments, the lysate solution is exposed to a substrate having a charged surface. Suitable substrates include, but are not limited to, ion resins, ceramics, mineralized tissues, demineralized tissues, soft tissues, metals, plastics, polymers, and combinations thereof. The surface of these substrates can inherently carry a charge or be configured such that they carry a charge. The surface of the substrate can carry a positive or negative charge. The charged surface of the substrate attracts oppositely charged intracellular components present in the lysate.

Continuing with FIG. 1, it is determined in step 102 if the lysate or separated intracellular components are to be neutralized or not. In some embodiments, the lysate or intracellular components are neutralized in step 103. In these embodiments, the pH of the lysate or a solution containing the separated desired intracellular components is adjusted to about 6 to about 8. In an embodiment, the pH of the lysate or the solution containing the separated desired intracellular components is adjusted to about 7. In one non-limiting example, HCL or acetic acid can optionally be used to render the solution more acidic or NaOH or a buffer (like PBS) may neutralize the solution or make it more basic.

In some embodiments, after neutralizing the lysate or the solution containing the separated desired intracellular components in step 103 or determining not to neutralize the lysate or the solution containing the separated desired intracellular components in step 102, it is determined in step 104 if the endogenous intracellular component slurry is to be stored or not. In embodiments where the endogenous intracellular component slurry is to be stored, the slurry is stored by a suitable method for later use in step 106. In some of these embodiments, the slurry is dehydrated (partial or complete). The dehydrated slurry can be cut to a desired shape and size. For example, the dehydrated slurry can be irregular, or about spherical, rectangular, triangular, or sheet-like. One of ordinary skill in the art will appreciate that the desired shape and size of the dehydrated slurry will depend on a variety of factors, including but not limited to, the implant use and the location of implantation. In other embodiments, the slurry is lyophilized. In some embodiments, the slurry, dehydrated slurry, or lyophilized slurry is placed in a suitable container. In some embodiments, the container is air tight. In other embodiments, the container can withstand freezing.

In some embodiments, the container contains information regarding the donor source, lot number, intracellular components contained therein, and/or other information, which identifies or otherwise characterizes the endogenous intracellular component slurry. In further embodiments, the slurry, dehydrated slurry, or lyophilized slurry is stored at about 4° C. to about −209° C. The slurry can be stored prior to use for up to about 5 years. In some embodiments, additional compounds are added to the slurry prior to storage. Suitable compounds include, but are not limited to, preservatives, cryoprotectants, diluents, antibiotics, antivirals, antifungals, pH stabilizers, osmostabilizers, protease inhibitors or combinations thereof.

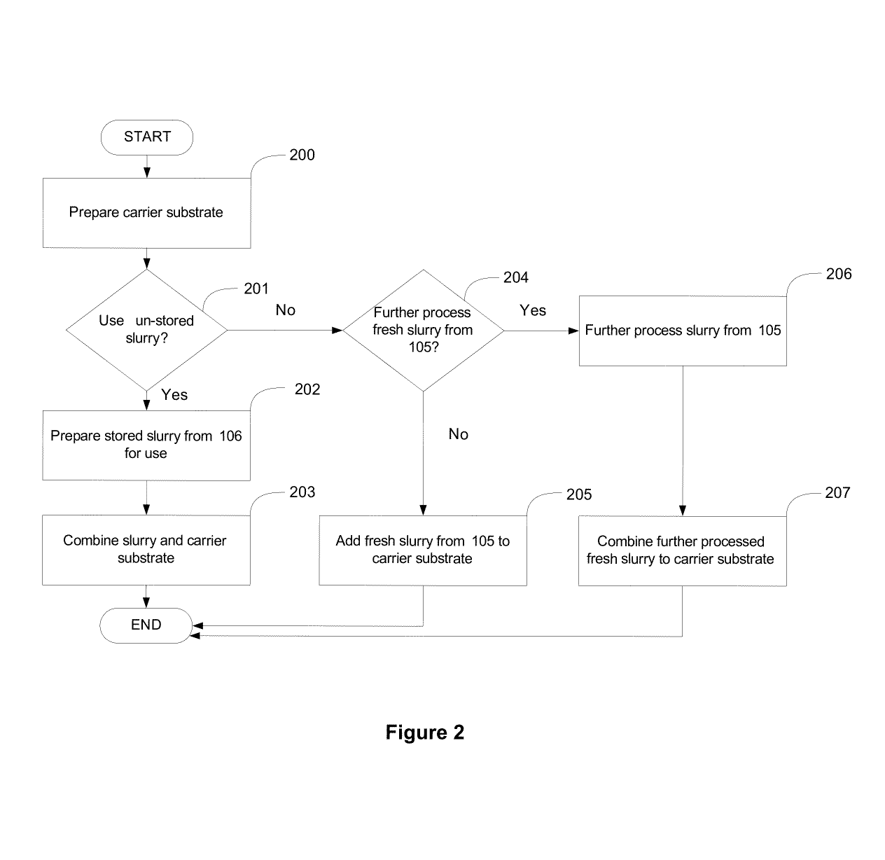

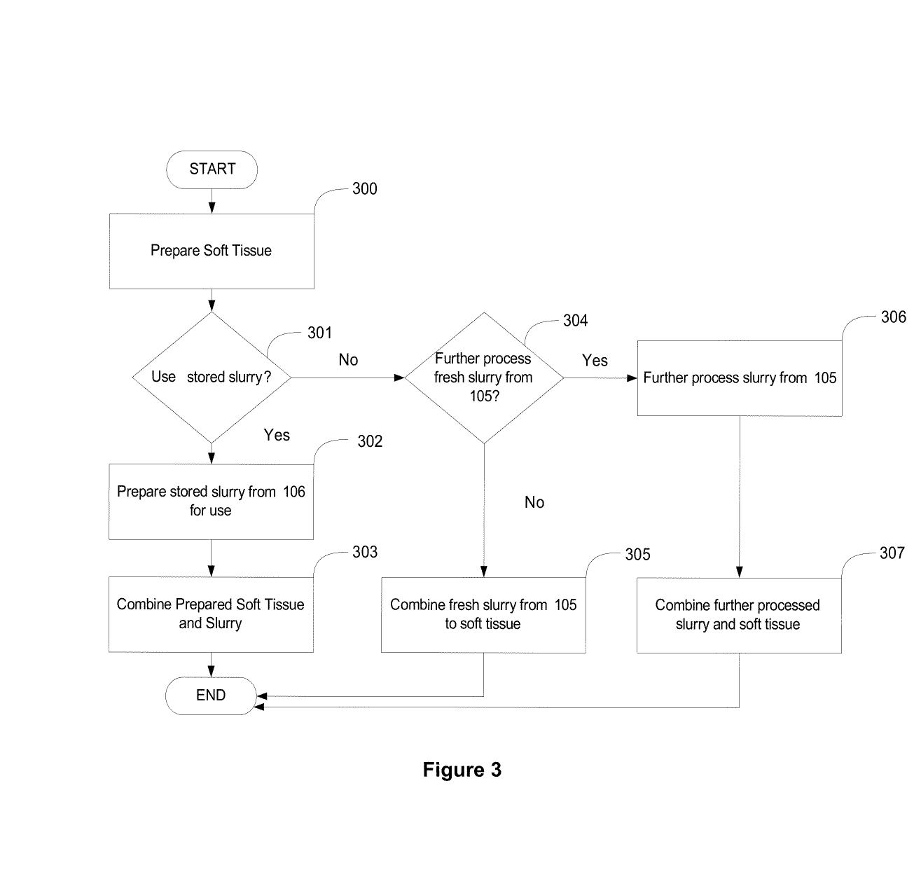

In some embodiments, it is determined in step 107 whether to use the stored slurry. In some embodiments where it is decided to use the stored slurry, the stored slurry is used in step 202 in FIG. 2. In other embodiments, the stored slurry is used in step 302 of FIG. 3.

In embodiments where it is determined in step 104 that the slurry is not to be stored, it is determined in step 105 whether to use the slurry containing endogenous intracellular components directly as filler for implantation in a subject. If it is decided to use the slurry directly as filler, the slurry is implanted into a subject as filler. In some embodiments, additional components are added to the slurry prior to use as a filler. Suitable compounds include, but are not limited to, preservatives, diluents, antibiotics, antivirals, antifungals, pH stabilizers, osmostabilizers, anti-inflammants, anti-neoplastics, chemotherapeutics, immunomodulators (including immunosuppressants), chemoattractants, growth factors, anticoagulants, or combinations thereof.

In some embodiments, the slurry is implanted into a subject at a location that has been determined by a medical practitioner to be in need of a filler. In addition to providing volume to the implantation site, the filler can aid in recruitment of compounds, such as growth factors and cytokines, to the implantation site. This facilitates the growth and development of existing cells and stimulates the growth and development of new cells at the implantation site. As such, when the filler is absorbed by the body, the subject's own cells remain in place to level out the depression in the skin. In one non-limiting example, a dermatologist or reconstructive medicine practitioner determines to use the filler to add substance to depressions in skin (e.g. wrinkles) to even out the skin surface and administers the filler to a depression in the skin.

In further embodiments, the filler is administered to a location in a subject that has a tissue implant graft already in place or is added to the site of a tissue graft during the same procedure that the tissue graft is being implanted in the subject. As previously described, the filler can aid in recruitment of compounds, such as growth factors and cytokines, to the implantation site. This facilitates the growth and developments of existing cells in the area and the growth and development of new cells at the implantation site. This process also enhances integration of the tissue graft to the surrounding tissue, which improves performance of the tissue graft.

In some embodiments where it is determined not to use the slurry as filler, the slurry can be used in steps 205 or 206 of FIG. 2. In other embodiments, the slurry can be used in steps 305 or 306 of FIG. 3. In some embodiments, prior to use in steps 205, 206, 305, or 306, additional compounds are added to the slurry. Suitable compounds include, but are not limited to, preservatives, diluents, antibiotics, antivirals, antifungals, pH stabilizers, osmostabilizers, anti-inflammants, anti-neoplastics, chemotherapeutics, immunomodulators (including immunosuppressants), chemoattractants, or combinations thereof.

During the generation of the slurry, the hydrophobic components of the adipose cells are separated from the hydrophilic components of the adipose cells. According to the steps previously described, the slurry contains only the hydrophilic components. However, in some embodiments, for example where increased lubricity is desired, the some of the hydrophobic components can be added back into the slurry.

Attention is now directed to FIG. 2, which is a flow diagram illustrating one embodiment of a method of incorporating the stored or un-stored slurry of FIG. 1 into a carrier substrate. As previously discussed, the slurry contains one or more intracellular components, which can enhance the performance of a soft tissue graft or implant. The embodiments discussed in relation to FIG. 2 are directed towards incorporating the intracellular components in a carrier substrate, which then can be administered to a subject in need thereof. In some embodiments, the carrier substrate is isolated along with the slurry. In other words, the slurry is generated such that it contains the carrier substrate as well as the intracellular growth factors and other hydrophilic components. In other embodiments, the slurry does not contain a carrier substrate. In either case, carrier substrate(s) can be added to the slurry as described below.

In some embodiments, the carrier substrate further enhances the performance of the soft tissue graft or implant. For example, the carrier substrate can be a scaffold, which provides an environment for cell growth and development. Suitable carrier substrates include but are not limited to, allogeneic, autologous, syngeneic, or xenogeneic complete extracellular matrix, decellularized extracellular matrix, or extracellular matrix components such as hydrogels, synthetic or natural polymer solids and semi-solids, carbohydrates, self-assembling peptides, carbon nanotubes, chitosan, alginate, hyaluronic acid, bone powder, cartilage powder, proteins, sugars, plastics, metals, or combinations thereof. In some embodiments, the carrier substrate is biocompatible. In embodiments, the carrier substrate is prepared for use 200 by methods generally known in the art. In some embodiments, the carrier substrate is already ready for use and no preparation is necessary. In some embodiments, the ratio of slurry to carrier substrate ranges from about 1:1 v/v to about 10:1 v/v. In other embodiments, the ratio of slurry to carrier substrate ranges from about 1:1 v/v to about 1:100 v/v.

After the carrier substrate is prepared 200, it is determined whether or not to use stored 106, (FIG. 1) or un-stored (fresh) 105, (FIG. 1) slurry 201. In embodiments where it is decided to use stored slurry, the stored slurry from step 106 (FIG. 1) is prepared for use in step 202. In some embodiments, preparation of the stored slurry includes thawing the slurry. In other embodiments, preparation of the stored slurry includes rehydrating the slurry. If the slurry is not rehydrated prior to use, it will become rehydrated upon introduction into the body of a subject when it contacts the biological fluids within the body. In further embodiments, the preparation process requires no additional preparation of the stored sample other than to take it from storage. After the stored slurry is prepared 202, the prepared slurry is then combined with the carrier substrate 203 using suitable methods.

In embodiments where it is decided to not to use the stored slurry, it is determined in step 204 whether to further process the fresh slurry from step 105 (FIG. 1). In embodiments where it is determined to further process fresh slurry from step 105 (FIG. 1), the slurry is further processed 206. The slurry can be further processed by filtering, concentrating, diluting, and/or fortifying with additional compounds, such as preservatives, antibiotics, antivirals, antifungals, pH stabilizers, osmostabilizers, anti-inflammants, anti-neoplastics, chemotherapeutics, immunomodulators (including immunosuppressants), chemoattractants, or combinations thereof.

After further processing 206, the further processed slurry is combined with the prepared carrier substrate 207. The carrier substrate containing the slurry can then be implanted into a subject in need thereof. In some embodiments, the carrier substrate containing the slurry is implanted into a subject at a location that has been determined by a medical practitioner to be in need thereof. In addition to providing volume to the implantation site, the carrier substrate containing the slurry can aid in recruitment of compounds, such as growth factors and cytokines, to the implantation site. This facilitates the growth and development of existing cells and stimulates the growth and development of new cells at the implantation site. As such, when the carrier substrate and/or slurry is absorbed by the body, the subject's own cells remain in place to level out the depression in the skin. In one non-limiting example, a dermatologist or reconstructive medicine practitioner determines to use the carrier substrate containing the slurry to add substance to depressions in skin (e.g. wrinkles) to even out the skin surface and administers the carrier substrate containing the slurry to a depression in the skin.

In further embodiments, the carrier substrate containing the slurry or components thereof is administered to a location in a subject that has a tissue implant already in place or is added to the site of a tissue graft during the same procedure that the tissue graft is being implanted in the subject. In other embodiments, the carrier substrate containing the slurry can be added to a tissue graft prior to the tissue graft from being implanted. As previously described, the carrier substrate containing the slurry can aid in recruitment of compounds, such as growth factors and cytokines, to the implantation site. This facilitates the growth and development of existing cells in the area and the growth and development of new cells at the implantation cite. This process also enhances integration of the tissue graft to the surrounding tissue, which improves performance of the tissue graft.

In embodiments where it is determined not to further process the fresh slurry from step 105 (FIG. 1), the fresh slurry is combined with the carrier substrate 205 as previously described. The combined carrier substrate/slurry can be administered to a subject in need thereof as previously described above with respect to processed fresh slurry.

Turning now to FIG. 3, which shows a flow diagram illustrating embodiments of a method of incorporating the stored or un-stored slurry of FIG. 1 into a soft tissue graft. As previously discussed, the slurry contains one or more intracellular components, which can enhance the performance of a soft tissue graft. The method begins with preparation of a soft tissue graft 300. In some embodiments, the soft tissue graft is harvested from a donor. The soft tissue graft can be allogeneic, autologous, syngeneic, or xenogeneic. In other embodiments, the soft tissue graft is obtained from a soft tissue graft developed or maintained by in vitro or ex vivo culture. In some embodiments, the soft tissue graft is cleaned, sterilized, and/or decellularized. In some embodiments, the soft tissue graft is ready to use and no preparation steps are needed.

After the soft tissue graft is prepared 300, it is determined whether or not to use stored 106, (FIG. 1) or un-stored (fresh) 105, (FIG. 1) slurry 201. In embodiments where it is decided to use stored slurry, the stored slurry from step 106 (FIG. 1) is prepared for use in step 302. In some embodiments, preparation of the stored slurry includes thawing the slurry. In other embodiments, preparation of the stored slurry includes rehydrating the slurry. If the slurry is not rehydrated prior to use, it will become rehydrated upon introduction into the body of a subject when it contacts the biological fluids within the body. In further embodiments, the preparation process requires no additional preparation of the stored sample other than to take it from storage.

After the stored slurry is prepared 302, the prepared slurry is combined with the soft tissue graft 303 using suitable methods. In some embodiments, the slurry is combined with the soft tissue graft prior to grafting the soft tissue graft in a subject. In other embodiments, the slurry is combined with the soft tissue graft after the soft tissue graft is already in place within a subject.

In embodiments where it is decided not to use stored slurry, it is determined whether or not to further process the fresh slurry from step 105 (FIG. 1). In embodiments where it is determined to further process fresh slurry from step 105 (FIG. 1), the slurry is further processed in step 306. The slurry can be further processed by filtering, concentrating, diluting, and/or fortifying with additional compounds, such as preservatives, antibiotics, antivirals, antifungals, pH stabilizers, osmostabilizers, anti-inflammants, anti-neoplastics, chemotherapeutics, immunomodulators (including immunosuppressants), angiogenic compounds, vasculogenic chemoattractants, or combinations thereof.

After further processing in step 306, the further processed slurry is combined with the prepared soft tissue graft in step 307. In some embodiments, the slurry is combined with the soft tissue graft prior to grafting the soft tissue graft in a subject. In other embodiments, the slurry is combined with the soft tissue graft after the soft tissue graft is already in place within a subject.

In embodiments where it is determined not to further process the fresh slurry from step 105, (FIG. 1), the fresh slurry is combined with the soft tissue graft 305. In some embodiments, the slurry is combined with the soft tissue graft prior to grafting the soft tissue graft in a subject. In other embodiments, the slurry is combined with the soft tissue graft after the soft tissue graft is already in place within a subject.

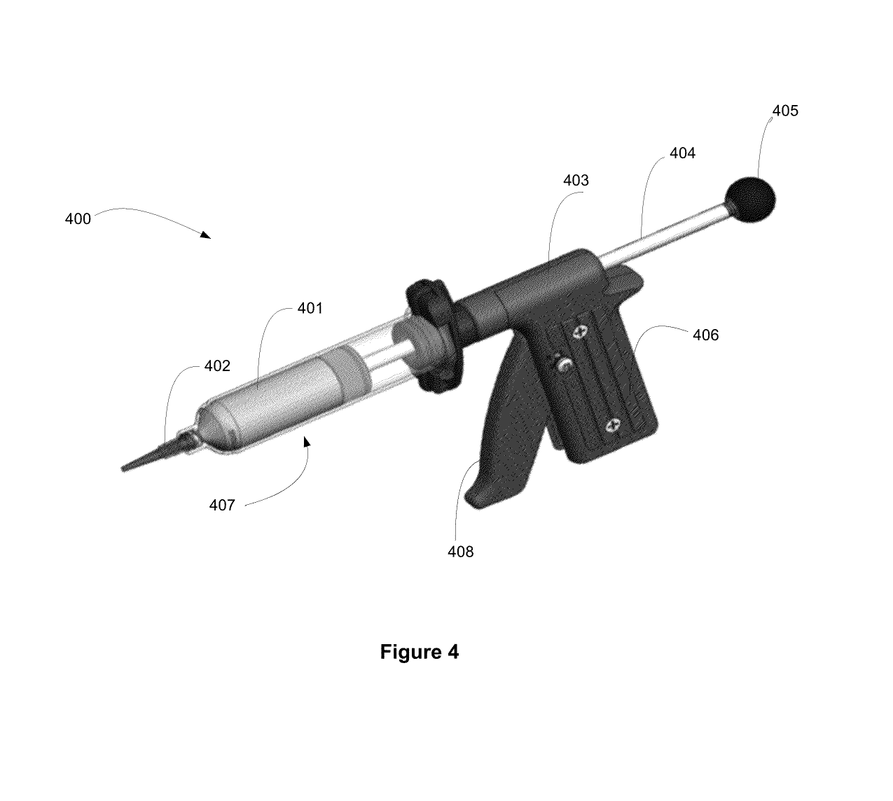

With embodiments of the methods of producing the slurry containing intracellular components, soft tissue implants and grafts combined with the slurry containing intracellular components understood, attention is directed to FIG. 4, which shows one embodiment of a delivery device 400 containing a slurry or combined slurry and carrier substrate 401, as produced according to the embodiments described herein. The delivery device 400 contains a tip 402 that is mechanically coupled to a hollow container 407. In some embodiments the tip 402 is tapered. The opening of the tip 402 can range from about 7 gauge to about 34 gauge. In some embodiments, the opening of the tip 402 is beveled. In other embodiments, the opening of the tip 402 is flush. In some embodiments, the tip 402 configured to mechanically lock onto the hollow container 407.

The hollow container 407 is configured to hold the slurry or the combined slurry and carrier substrate 401. In some embodiments, the hollow container 407 is configured to hold about 0.1 cc to about 1000 cc of slurry or the slurry combined with a carrier substrate. In one embodiment, the hollow container 407 is configured to hold up to about 1 cc of slurry or slurry/carrier substrate mixture. In another embodiment, the hollow container 407 is configured to hold up to about 5 cc of slurry or slurry/carrier substrate mixture. In yet further embodiments, the hollow container 407 is configured to hold up to about 10 cc of slurry or slurry/carrier substrate mixture. In yet further embodiments, the hollow container 407 is configured to hold up to about 20 cc of slurry or slurry/carrier substrate mixture. In other embodiments, the hollow container 407 is configured to hold up to about 50 cc of slurry or slurry/carrier substrate mixture. In still other embodiments, the hollow container 407 is configured to hold up to about 100 cc of slurry or slurry/carrier substrate mixture. In further embodiments, the hollow container 407 is configured to hold up to about 500 cc of slurry or slurry/carrier substrate mixture. In other embodiments, the hollow container 407 is configured to hold up to about 1000 cc of slurry or slurry/carrier substrate mixture.

In an embodiment, the hollow container is coupled to a handle 403 that is made up of a first grip 406 and a trigger portion 402. A movable plunger 404 is mechanically coupled to the handle 403 and hollow container 407. The movable plunger 404 extends through the handle 403 and into the end of the hollow container 407 opposite of the tip 402. The movable plunger 404 is configured to apply positive or negative pressure to the hollow container and the contents contained therein. At the end opposite the hollow container, the movable plunger contains a second grip 405.

In some embodiments, positive pressure is applied to the hollow container by applying pressure on the second grip 405 and pushing the second grip 405 towards the handle 403. In other embodiments, the trigger 408 is squeezed. The trigger 408 is configured such that it applies a positive pressure on the plunger when the trigger 408 is squeezed. When pressure is applied to the second grip 405 or trigger 408, and the plunger end inside the hollow container 407 moves closer to the tip 402, this expels the slurry or combined slurry and carrier substrate 401 from the device 400. Negative pressure is applied by pulling on the second grip 405 and pulling the second grip 405 away from the handle 403. This moves the end of the movable plunger 404 that is inside the hollow container 407 closer to the handle 403 and away from the tip 402. Negative pressure pulls content into the hollow container 407. In further embodiments, the delivery device 400 is configured such that positive or negative pressure is generated by a machine as opposed to a human user.

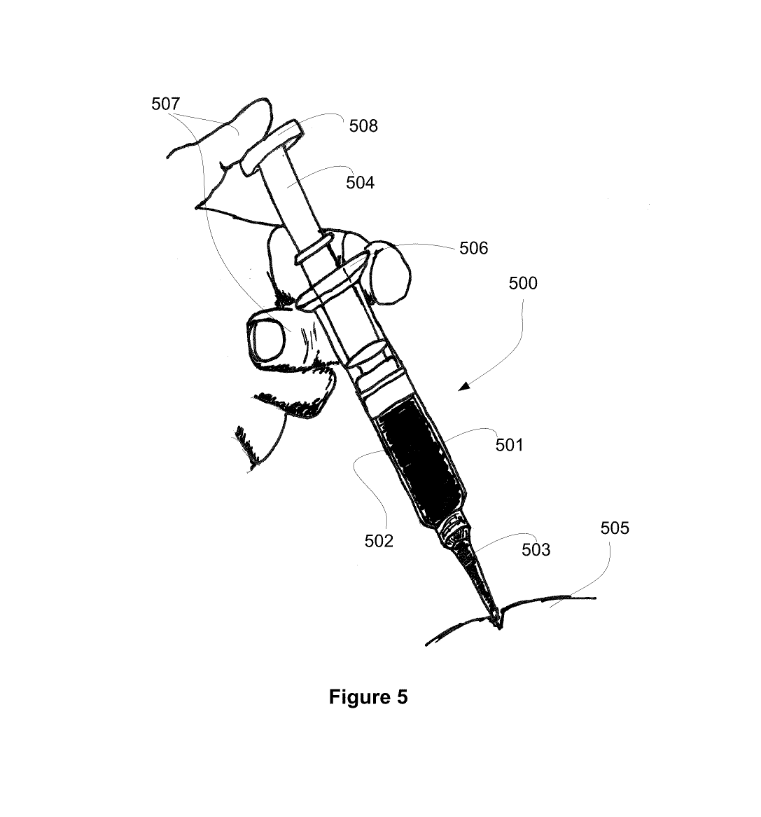

FIG. 5 shows another embodiment of a delivery device 500 containing a slurry or combined slurry and carrier substrate 501 as produced according to the methods described herein. The delivery device 500 contains a tip 503 that is mechanically coupled to a hollow container 502. In some embodiments, the tip 503 is tapered. The opening of the tip 503 can range from about 7 gauge to about 34 gauge. In some embodiments, the opening of the tip 503 is beveled. In other embodiments, the opening of the tip 503 is flush. In some embodiments, the tip 503 configured to mechanically lock onto the hollow container 503. For example, the mechanical lock can be a luer lock.

The hollow container 502 is configured to hold the slurry or the combined slurry and carrier substrate 501. In some embodiments, the hollow container 502 is coupled to a ridge portion 506 that forms a grip for fingers of a user 507 as shown in FIG. 5. A movable plunger 504 is mechanically coupled to the hollow container 502. The movable plunger 504 extends through one end of the hollow container 502 opposite of the tip 503. The movable plunger 504 is configured to apply positive or negative pressure to the hollow container 502 and the contents contained therein. At the end opposite to the hollow container 502, the movable plunger 504 contains a thumb rest 508.

In one embodiment, positive pressure is applied to the hollow container 502 by pressure to the thumb rest 508, and thus, depresses the plunger 504 further into the hollow container 502. In some embodiments, a user holds the device 500 between two or more fingers 507. One finger 507, for example the thumb, can be placed on the thumb rest 508, while one or more other fingers 507 can be placed on either side of the hollow container 502 under the ridge portion 506, as demonstrated in FIG. 5. Positive pressure can be applied to the hollow container 502 by moving the thumb 507 closer to the other finger(s) 507 under the ridge portion 506. This depresses the plunger 504 and creates positive pressure on the hollow container 502. Negative pressure can be applied by pulling back on the plunger 504. Positive pressure expels contents 501 of the hollow container 502 and negative pressure draws contents into the hollow container 502. In some embodiments, the application of positive pressure expels the contents 501 of the hollow container 502 into a subject in need thereof 505. In further embodiments, the delivery device 500 is configured such that positive or negative pressure is generated by a machine as opposed to a human user. For example, in some embodiments the delivery device 500 is loaded into a machine, which contains portion, which applies positive pressure to the movable plunger 504. Examples of such machines are known in the art.

Also provided herein are soft tissue implants that contain a bioactive intracellular component of an adipose cell. In some embodiments, the soft tissue implant is a slurry. In one embodiment, the slurry is derived from adipocytes that are harvested from in vitro cultured adipocytes or from adipocytes harvested directly from tissue. In other embodiments, the slurry is derived from other types of soft tissue cells. Such cells include, but are not limited to, muscle, epithelial cells, tendons, and ligaments. The intracellular components contained in the slurry include but are not limited to proteins (both structural and non-structural), nucleic acids, lipids, carbohydrates, and other molecules. In some embodiments, the slurry contains an enriched or concentrated amount of these endogenous intracellular components. In some embodiments, the donor cells are selectively lysed, as previously described, such that the slurry selectively contains growth factors, particularly vascular endothelial growth factor (VEGF), basic fibroblast growth factor (bFGF), transforming growth factor beta 1 (TGFb1), acidic fibroblast growth factor (aFGF), insulin-like growth factor (IGF).

As previously discussed, an effective amount of the slurry prepared according to the methods described herein, can be administered to subjects in need thereof as a filler. In some embodiments, the slurry is configured as a paste. In other embodiments, an effective amount of the slurry can already contain and/or be combined with a carrier substrate as previously described, and the combination can then be administered to a subject in need thereof. In further embodiments, an effective amount of the slurry can be administered after placement of a soft tissue graft (other than one already incorporating the slurry). In other embodiments, an effective amount of the slurry can be incorporated directly to a soft tissue graft (that is not the slurry or slurry/carrier substrate itself) ex vivo prior to implantation. The effective dose may be between about 1 mL to 1000 ml.

The slurries (including those containing a carrier substrate), implants, and grafts and delivery devices described herein can be presented as a combination kit. As used herein, the terms “combination kit” or “kit of parts” refers to the slurries, implants, and grafts and delivery devices and additional components that are used to package, sell, market, deliver, and/or administer the combination of elements or a single element, such as the active ingredient, contained therein. Such additional components include but are not limited to, packaging, syringes, blister packages, bottles, and the like. In one embodiment the kit contains a soft tissue implant containing a bioactive intracellular component of an adipose cell, and a carrier substrate. In some embodiments, the soft tissue implant contained in the kit is generated by a method involving harvesting an adipose cell from a donor, selectively lysing the adipose cell to obtain a bioactive intracellular component, and combining the bioactive intracellular component with a carrier substrate.

In some embodiments, the combination kit also includes instructions printed on or otherwise contained in a tangible medium of expression. The instructions can provide information regarding the content of the compound or pharmaceutical formulations contained therein, safety information regarding the content of the slurry(ies), implant(s), graft(s), and delivery device(s) contained therein, information regarding the dosages, indications for use, and/or recommended treatment regimen(s) for the slurry(ies), implant(s), graft(s), and delivery device(s) contained therein. In an embodiment, the instructions provide directions for administering the soft tissue implant to a subject in need thereof as a filler or as part of a tissue graft being implanted in the subject. In some embodiments, the instructions provide directions for administering the slurry(ies), implant(s), and graft(s) to a subject in need thereof. Indications for use include, but are not limited to, reduction of fibrous capsule formation after other soft tissue implants (e.g. soft tissue (i.e., breast), vascular (i.e. stents), or joint implants) caused by the introduction of allogeneic cells or other foreign bodies, reduction of implant induced inflammation, improving implant integration into surrounding tissue, improving quality or coloring of skin, or repair of depressions in skin or other soft tissue.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Increased Growth Factors in Soft Tissue Implants Containing Adipose-Derived Intracellular Compounds

Introduction

Soft tissue implants made according to the methods described herein contain intracellular components, including growth factors such as vascular endothelial growth factor (VEGF), basic fibroblast growth factor (bFGF), and transforming growth factor beta 1 (TGFb1). In order to assess the growth factor content of the soft tissue implants described herein, adipose derived intracellular content was harvested and processed according to methods described herein and applied to an extracellular matrix. This composition is referred to as LipoAmp in this Example. The growth factor content of LipoAmp was compared to a control soft tissue implant as described in Brown, et al. 2011. Tissue Eng. Part C, 17:411-423.

Materials and Methods

Briefly, subcutaneous fat was separated from the dermal layer of a subject. The harvested subcutaneous fat was ground via a blender to mechanically disrupt the cellular structure to form a mixture of hydrophilic and hydrophobic components. The hydrophilic and hydrophobic components were separated from one another based on their buoyancy. The hydrophobic portion, which contains inter alia the lipids, was discarded. Acetic acid (up to 50% v/v, e.g. about 25% v/v) was added to the hydrophilic fraction. The optional step of adding up to 1M HCl, was performed. Here, 0.6N HCl was added to the hydrophilic fraction. The resulting solution was then neutralized in phosphate buffered saline or NaOH as necessary. Excess liquids were removed via centrifugations.

Results

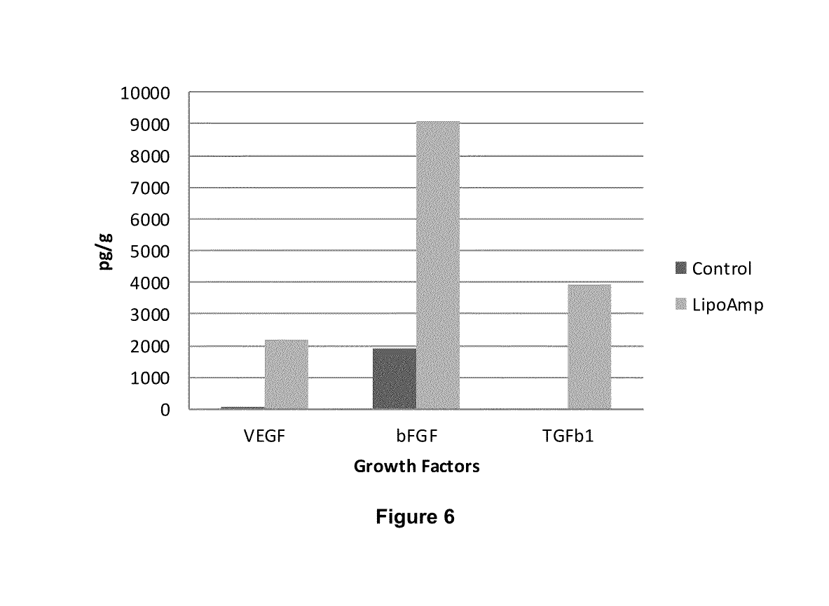

The results of this experiment are shown in FIG. 6, which demonstrates increased growth factor content in a carrier substrate combined with adipose-derived intracellular compounds (“LipoAmp”) as compared to control. Concentration (pg/g of implant) of the growth factors is shown on the y axis. The growth factors are shown on the x-axis. The soft tissue implant composition as described herein had a greater amount of VEGF, bFGF, and TGFb1.

### Example 2

### Increased Adipose-Derived Soft Tissue Implantation Volume Compared to Native Tissue in Vivo

Introduction

The effect of a soft tissue implant made and administered according to the methods described herein (“LipoAmp”) on implant volume post implantation was examined in vivo.

Materials and Methods

LipoAmp was prepared as previously described in Example 1.

Results

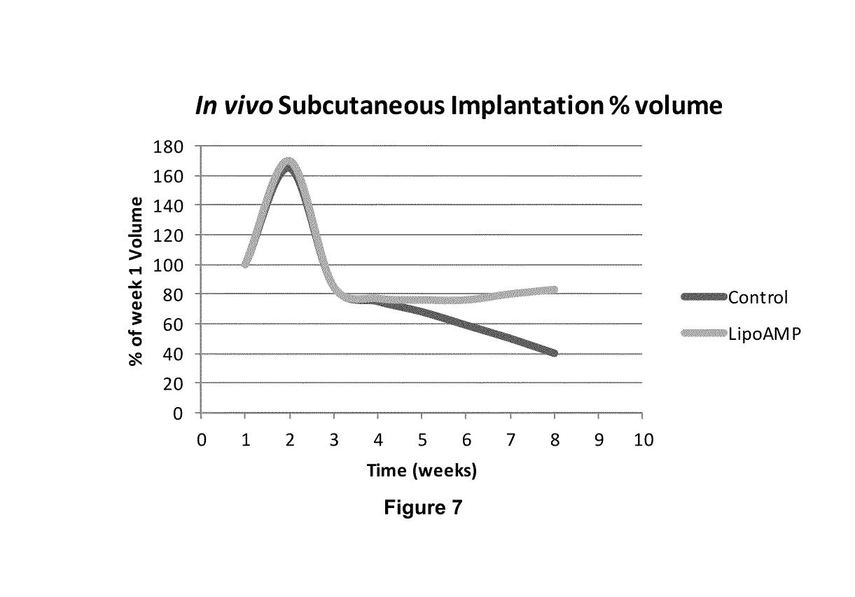

The results of this experiment are demonstrated in FIG. 7. As demonstrated by FIG. 7, while the Lipoamp implant and control maintained about the same volume, at about week 4, the performance of the two implants diverged. Over weeks 5 to 8, the Lipoamp implant maintained the volume at approximately 8 percent of the volume present at the start of the experiment. In contrast, the control implant decreased steadily in volume over weeks 5 to 8.

### Example 3

### Soft Tissue Implant Containing Adipose-Derived Intracellular Compounds Induces Ectopic Adipogenesis in Vivo

Introduction

The effect of a soft tissue implant made and administered according to methods described herein (“LipoAmp”) on adipogenesis was examined in vivo.

Materials and Methods

To generate the LipoAmp, subcutaneous fat was separated from the dermal layer of a subject. The harvested subcutaneous fat was ground via a blender to mechanically disrupt the cellular structure to form a mixture of hydrophilic and hydrophobic components. The hydrophilic and hydrophobic components were separated from one another based on their buoyancy. The hydrophobic portion, which contains inter alia the lipids, was discarded. Acetic acid (up to 50% v/v, e.g. about 25% v/v) was added to the hydrophilic fraction. The optional step of adding up to 1M HCl, was performed. Here, 0.6N HCl was added to the hydrophilic fraction. The resulting solution was then neutralized in phosphate buffered saline or NaOH as necessary. Excess liquids were removed via centrifugations. The LipoAmp was then administered to a subject.

Results

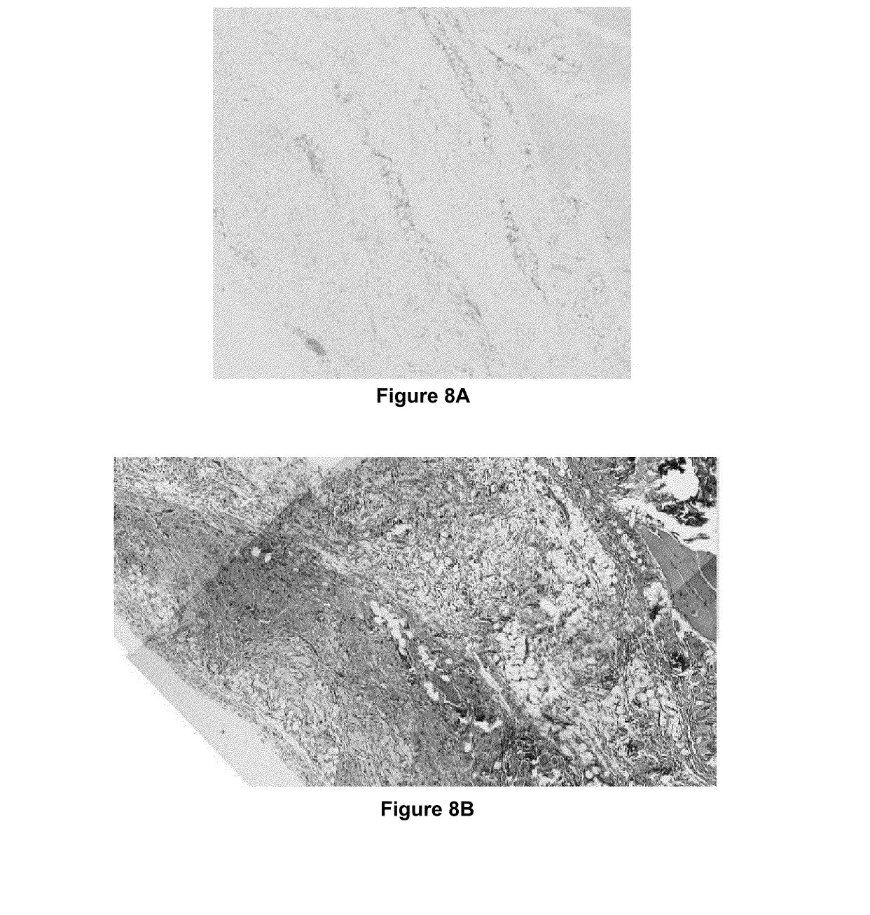

The results of this experiment are shown in FIGS. 8A and 8B. As demonstrated in FIG. 8B, adipogenesis is induced from the implant.

We claim:

1. A soft tissue implant comprising:
a bioactive intracellular component of an adipose cell; and
a carrier substrate, where the soft tissue implant is prepared by a method comprising:
harvesting an adipose cell from a donor;
selectively lysing the adipose cell to obtain a bioactive intracellular component; and
combining the bioactive intracellular component with a carrier substrate.

2. The soft tissue implant of claim 1, wherein the bioactive intracellular component is a growth factor.

3. The soft tissue implant of claim 1, wherein the donor is selected from the group consisting of an autologous donor, allogeneic donor, xenogeneic donor, and a syngeneic donor.

4. The soft tissue implant of claim 1, wherein the step of selectively lysing further comprises selectively lysing the adipose cell by chemical disruption or mechanical disruption.

5. The soft tissue implant of claim 4, wherein the chemical disruption comprises contacting the adipose cell with a solution, the solution comprising an acid or a base.

6. The soft tissue implant of claim 1, wherein the step of selectively lysing further comprises selective separation of the bioactive intracellular component from other adipose cell components.

7. The soft tissue implant of claim 1, wherein the carrier substrate is selected from the group consisting of a complete extracellular matrix, a decellularized extracellular matrix, extracellular matrix components, a hydrogel, a polymer solid, a polymer semi-solid, a carbohydrate, self-assembling peptides, carbon nanotubes, chitosan, alginate, hyaluronic acid, bone powder, cartilage powder, a protein, a sugars, a plastic, a metal, and combinations thereof.

8. The soft tissue implant of claim 1, wherein the bioactive intracellular content is contained in a slurry, and wherein a ratio of slurry to carrier substrate is about 1:1 (v/v) to about 1:100 (v/v).

9. A method comprising:
harvesting an adipose cell from a donor;
selectively lysing the adipose cell to obtain a bioactive intracellular component; and
combining the bioactive intracellular component with a carrier substrate to form a combined bioactive intracellular component-carrier substrate.

10. The method of claim 9, wherein the bioactive intracellular component is a growth factor.

11. The method of claim 9, wherein the donor is selected from the group consisting of an autologous donor, allogeneic donor, xenogeneic donor, and a syngeneic donor.

12. The method of claim 9, wherein the step of selectively lysing further comprises selectively lysing the adipose solution by chemical disruption or mechanical disruption.

13. The method of claim 12, wherein the chemical disruption comprises contacting the adipose cell with a solution, the solution comprising an acid or a base.

14. The method of claim 9, wherein the step of selectively lysing further comprises selective separation of the bioactive intracellular component from other adipose cell components.

15. The method of claim 9, wherein the carrier substrate is selected from the group consisting of a complete extracellular matrix, a decellularized extracellular matrix, extracellular matrix components, a hydrogel, a polymer solid, a polymer semi-solid, a carbohydrate, self-assembling peptides, carbon nanotubes, chitosan, alginate, bone powder, cartilage powder, a protein, a sugars, a plastic, a metal, and combinations thereof.

16. The method of claim 9, wherein the wherein the bioactive intracellular content is contained in a slurry, and wherein the slurry ratio of slurry to carrier substrate is about 1:1 (v/v) to about 1:100 (v/v).

17. The method of claim 9, further comprising adding a compound from the group consisting of: preservatives, antibiotics, antivirals, antifungals, pH stabilizers, osmostabilizers, anti-inflammants, anti-neoplastics, growth factors, angiogenic compounds, vasculogenic compounds, chemotherapeutics, immunomodulators, chemoattractants, and combinations thereof to the intracellular component, the carrier substrate or the combined bioactive intracellular component-carrier substrate.

18. The method of claim 9, further comprising administering the combined bioactive intracellular component-carrier substrate to a subject in need thereof.

19. A kit comprising:
a soft tissue implant comprising:
a bioactive intracellular component of an adipose cell; and
a carrier substrate, where the soft tissue implant is generated by a method comprising:
harvesting an adipose cell from a donor;
selectively lysing the adipose cell to obtain a bioactive intracellular component; and
combining the bioactive intracellular component with a carrier substrate;
and
instructions contained in a tangible medium of expression, wherein the instructions provide directions for administering the soft tissue implant into a subject in need thereof.

20. The kit of claim 19, further comprising a delivery device having a hollow container and a plunger, wherein the plunger is mechanically coupled to the hollow container, and wherein the delivery device is configured to contain the soft tissue implant within the hollow container.
